(19) United States
(12) Patent Application Publication
(10) Pub. No.: US 2021/0313070 A1
(43) Pub. Date: Oct. 7, 2021

(54) DEMENTIA PREDICTION DEVICE, PREDICTION MODEL GENERATION DEVICE, AND DEMENTIA PREDICTION PROGRAM

(71) Applicants: FRONTEO, Inc., Tokyo (JP); Keio Univesity, Tokyo (JP)

(72) Inventors: Hiroyoshi TOYOSHIBA, Tokyo (JP); Hidefumi UCHIYAMA, Tokyo (JP); Taishiro KISHIMOTO, Tokyo (JP); Kei FUNAKI, Tokyo (JP); Yoko SUGA, Tokyo (JP); Shogo HOTTA, Tokyo (JP); Takanori FUJITA, Tokyo (JP); Masaru MIMURA, Tokyo (JP)

(21) Appl. No.: 17/271,379

(22) PCT Filed: Jul. 3, 2019
(86) PCT No.: PCT/JP2019/026484

§ 371 Date: Feb. 25, 2021

(30) Foreign Application Priority Data
Sep. 12, 2018 (JP) 2017-170847

(51) Int. Cl. G16H 50/30; G06N 20/00; G06N 5/04; G16H 50/20; G16H 50/70; G06F 40/268; G06F 40/284; G06F 40/35; G06F 40/253; A61B 5/00

(52) U.S. Cl. CPC G16H 50/30; G06N 20/00; G06N 5/04; G16H 50/20; G16H 50/70; A61B 5/7267; G06F 40/284; G06F 40/35; G06F 40/253; A61B 5/4088; A61B 5/7275; G06F 40/268

(57) ABSTRACT

A relationship index value computation unit 100A that extracts n words from m texts representing contents of free conversations conducted by m patients whose severity of dementia is known, and computes a relationship index value reflecting a relationship between the m texts and the n words, a prediction model generation unit 14A that generates a prediction model for predicting severity of dementia based on a text index value group including n relationship index values for one text, and a dementia prediction unit 21A that predicts severity of dementia of a patient from a text subjected to prediction by applying the relationship index value computed by the relationship index value computation unit 100A from a text input by a prediction data input unit 20 to a prediction model are included, and severity of dementia can be predicted without performing a mini-mental state examination.

### TECHNICAL FIELD

The present invention relates to a dementia prediction device, a prediction model generation device, and a dementia prediction program, and particularly relates to a technology for predicting the severity of dementia of a patient (including a possibility that the patient has dementia), and a technology for generating a prediction model used for this prediction.

### BACKGROUND ART

Dementia continues to increase with the aging of the population, and has become a major social issue as well as a medical problem. Early detection and evaluation of severity of dementia are significantly important in the treatment of dementia. Currently, the mini-mental state examination (MMSE) has been widely used in daily clinical practice for screening tests for dementia and evaluation of severity. The MMSE is a cognitive function test including 11 items and 30 points of questions for examining insight, memory, attention (calculation), linguistic ability, composition ability (graphical ability), etc. Of the 30 points, 27 points or less is suspected of mild cognitive impairment (MCI), and 23 points or less is suspected of dementia.

Conventionally, there has been known a system in which evaluation is performed for each evaluation item of the MMSE to determine a possibility of developing dementia, and nursing care support is provided based on a determination result (for example, see Patent Document 1). In the system described in Patent Document 1, a physical or mental health condition of a care recipient is investigated by MMSE, and the health condition of the care recipient is evaluated from an investigation result. Then, audio or video is created according to the evaluation of the health condition of the care recipient and distributed to a caregiver, and the caregiver cares for the care recipient based on the distributed audio or video. Thereafter, the physical or mental health condition of the care recipient is re-examined and the health condition of the care recipient is re-investigated. It is stated that the investigation is conducted from the perspectives of four items of memory impairment, insight, activities of daily living (ADL), and physical function.

### CITATION LIST

### Patent Document

Patent Document 1: JP-A-2002-251467

### SUMMARY OF THE INVENTION

### Technical Problem

The MMSE has been widely known as a highly reproducible test. However, when the same patient is tested a plurality of times, a practice effect causes the patient to memorize the content of the question, making it impossible to measure an accurate score. Therefore, there is a problem that it is difficult to frequently measure the severity of dementia. The system described in Patent Document 1 described above does not take into consideration the problem that the MMSE is unsuitable for repeated use.

The invention has been made to solve such a problem, and an object of the invention is to obtain a measurement result excluding a practice effect by a patient even when the severity of dementia is repeatedly measured.

### Solution to Problem

To solve the above-mentioned problem, in a dementia prediction device of the invention, a plurality of texts representing contents of free conversations conducted by a plurality of patients whose severity of dementia is known, respectively, is input as learning data, morphemes of the plurality of input texts are analyzed to extract a plurality of decomposition elements, each of the plurality of texts is converted into a q-dimensional vector according to a predetermined rule, thereby computing a plurality of text vectors including q axis components, and each of the plurality of decomposition elements is converted into a q-dimensional vector according to a predetermined rule, thereby computing a plurality of element vectors including q axis components. Further, each of inner products of the plurality of text vectors and the plurality of element vectors is obtained, thereby computing a relationship index value reflecting a relationship between the plurality of texts and the plurality of decomposition elements. Then, a prediction model for predicting severity of dementia is generated based on a text index value group including a plurality of relationship index values for one text. When severity of dementia is predicted for a patient subjected to prediction, a text representing content of a free conversation conducted by the patient subjected to prediction is input as prediction data, and a relationship index value obtained by executing respective processes of element extraction, text vector computation, element vector computation, and index value computation on the input prediction data are applied to a prediction model, thereby predicting the severity of the dementia of the patient subjected to prediction.

### Advantageous Effects of the Invention

According to the invention configured as described above, since severity of dementia is predicted by analyzing a free conversation conducted by a patient, there is no need to perform a mini-mental state examination (MMSE). For this reason, even when the severity of the dementia is repeatedly measured, it is possible to obtain a measurement result (prediction result) excluding a practice effect by a patient. In particular, when a patient suffers from dementia, a conversational characteristic peculiar to dementia can be seen in a free conversation, a relationship index value is computed in a state where such a conversational characteristic is reflected, and a prediction model is generated using the relationship index value. Thus, it is possible to predict severity of the dementia from the free conversation conducted by the patient.

### BRIEF DESCRIPTION OF THE DRAWINGS

FIG. 1 is a block diagram illustrating a functional configuration example of a dementia prediction device according to a first embodiment.

FIG. 2 is an explanatory diagram of a text index value group according to the first embodiment.

FIG. 3 is a flowchart illustrating an operation example of the dementia prediction device according to the first embodiment.

FIG. 4 is a block diagram illustrating a functional configuration example of a dementia prediction device according to a second embodiment.

FIG. 5 is a diagram illustrating processing content of a part-of-speech extraction unit according to the second embodiment.

FIG. 6 is a diagram showing an example of a part of speech extracted by the part-of-speech extraction unit according to the second embodiment.

FIG. 7 is a block diagram illustrating a functional configuration example of a dementia prediction device according to a third embodiment.

FIG. 8 is a diagram illustrating processing content of a prediction model generation unit according to the third embodiment.

FIG. 9 is a block diagram illustrating a functional configuration example of a dementia prediction device according to a fourth embodiment.

FIGS. 10A and 10B are block diagrams illustrating a functional configuration example of the dementia prediction device according to the fourth embodiment.

FIG. 11 is a block diagram illustrating a functional configuration example of a dementia prediction device according to a fifth embodiment.

FIG. 12 is a block diagram illustrating a modification of the dementia prediction device.

### MODE FOR CARRYING OUT THE INVENTION

### First Embodiment

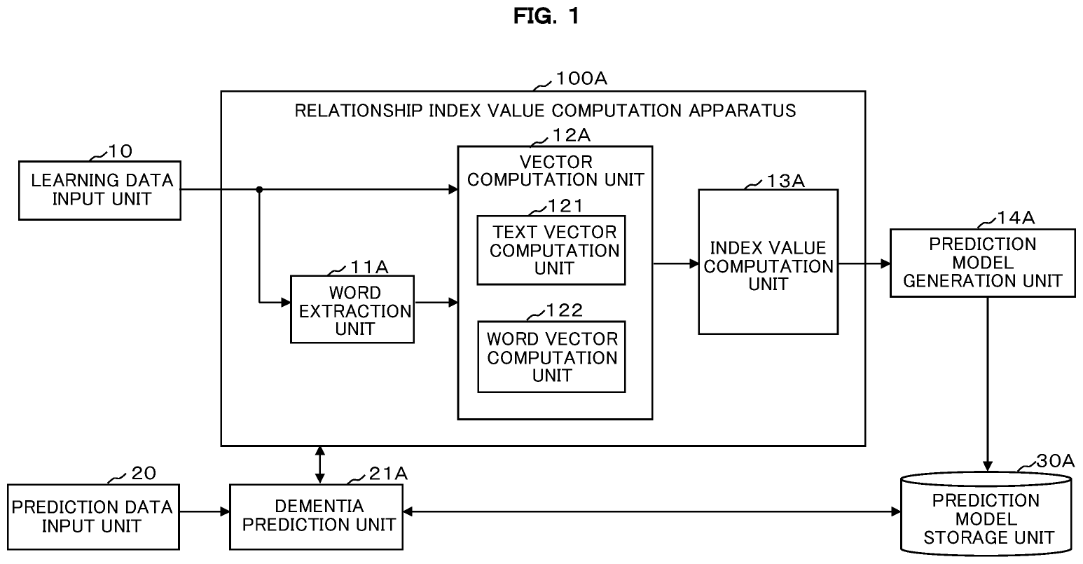

Hereinafter, a first embodiment according to the invention will be described with reference to the drawings. FIG. 1 is a block diagram illustrating a functional configuration example of a dementia prediction device according to the first embodiment. The dementia prediction device according to the first embodiment includes a learning data input unit 10, a word extraction unit 11A, a vector computation unit 12A, an index value computation unit 13A, a prediction model generation unit 14A, a prediction data input unit 20, and a dementia prediction unit 21A as a functional configuration. The vector computation unit 12A includes a text vector computation unit 121 and a word vector computation unit 122 as a more specific functional configuration. In addition, the dementia prediction device of the present embodiment includes a prediction model storage unit 30A as a storage medium. Note that for convenience of explanation, a part including the word extraction unit 11A, the vector computation unit 12A, and the index value computation unit 13A is referred to as a relationship index value computation unit 100A.

The relationship index value computation unit 100A inputs text data related to a text, and computes and outputs a relationship index value reflecting a relationship between the text and a word contained therein. In addition, in the dementia prediction device of the present embodiment, the relationship index value computation unit 100A analyzes a text representing content of a free conversation conducted by a patient, and severity of dementia of the patient is predicted from the content of the free conversation by the patient using a relationship index value computed by the analysis. A prediction model generation device of the invention includes the learning data input unit 10, the relationship index value computation unit 100A, and the prediction model generation unit 14A.

In the present specification, the term “text” generally refers to a text including two or more sentences divided by a period. In particular, in the present specification, a plurality of remark contents (corresponding to a plurality of sentences) spoken by a patient in a series of free conversations (continuous dialogue) conducted between a doctor and the patient is collectively treated as one text. That is, one text including a plurality of sentences is defined for one free conversation (a series of dialogues) of one patient.

Each of the functional blocks illustrated in FIG. 1 can be configured by any of hardware, a Digital Signal Processor (DSP), and software. For example, in the case of being configured by software, each of the functional blocks actually includes a CPU, a RAM, a ROM, etc. of a computer, and is implemented by operation of a program stored in a recording medium such as a RAM, a ROM, a hard disk, or a semiconductor memory.

The learning data input unit 10 inputs, as learning data, m texts representing contents of free conversations conducted by m patients (m is an arbitrary integer of 2 or more) whose severity of dementia is known, respectively. For example, the learning data input unit 10 replaces voice of a free conversation conducted between a patient and a doctor given an MMSE score by a pre-trained doctor with character data, and inputs a text of an utterance part of the patient included in the character data as learning data. In this case, the known severity of dementia for the patient means a value of the MMSE score. The learning data input unit 10 inputs m texts acquired from free conversations of m patients, respectively, as a plurality of pieces of learning data.

For example, the free conversation between the patient and the doctor is conducted in the form of an interview for 5 to 10 minutes. That is, a dialogue in the form in which the doctor asks the patient a question, and the patient answers the question is repeatedly conducted. Then, the dialogue at this time is input from a microphone and recorded, and voice of a series of dialogues (free conversations) is replaced with character data by manual transcription or using automatic voice recognition technology. From this character data, only the utterance part by the patient is extracted and used as learning data. Note that when the voice of the free conversation is replaced with the character data, only the utterance part by the patient may be replaced with the character data.

The word extraction unit 11A is an example of an “element extraction unit” in the claims, which analyzes m texts input as learning data by the learning data input unit 10, and extracts n words (n is an arbitrary integer of 2 or more) (corresponding to a decomposition element in the claims) from the m texts. As a method of analyzing texts, for example, a known morphological analysis can be used. Here, the word extraction unit 11A may extract morphemes of all parts of speech divided by the morphological analysis as words, or may extract only morphemes of a specific part of speech as words.

Note that the same word may be included in the m texts a plurality of times. In this case, the word extraction unit 11A does not extract the plurality of the same words, and extracts only one. That is, the n words extracted by the word extraction unit 11A refer to n types of words. However, each of the extracted n words is accompanied by information indicating an appearance frequency in the text. Here, the word extraction unit 11A may measure a frequency at which the same word is extracted from the m texts, and extract n (n types of) words from the one having the highest appearance frequency, or n (n types of) words having the appearance frequency equal to or higher than a threshold value.

A patient suffering from dementia has a tendency to repeat words spoken by the patient once many times. In addition, the patient suffering from dementia is less likely to speak spontaneously and may have a tendency to repeat conversations (echolalia) in which similar words are repeated in response to questions from doctors. Therefore, the word extraction unit 11A extracts n words from a text of a free conversation including a conversational characteristic peculiar to dementia.

The vector computation unit 12A computes m text vectors and n word vectors from the m texts and the n words. Here, the text vector computation unit 121 converts each of the m texts to be analyzed by the word extraction unit 11A into a q-dimensional vector (q is an arbitrary integer of 2 or more) according to a predetermined rule, thereby computing the m text vectors including q axis components. In addition, the word vector computation unit 122 converts each of the n words extracted by the word extraction unit 11A into a q-dimensional vector according to a predetermined rule, thereby computing the n word vectors including q axis components.

In the present embodiment, as an example, a text vector and a word vector are computed as follows. Now, a set S=<d ∈ D, w ∈ W> including the m texts and the n words is considered. Here, a text vector dᵢ→ and a word vector wⱼ→ (hereinafter, the symbol “→” indicates a vector) are associated with each text dᵢ (i=1, 2, . . . , m) and each word wⱼ (j=1, 2, . . . , n), respectively. Then, a probability P(wⱼ|dᵢ) shown in the following Equation (1) is calculated with respect to an arbitrary word w and an arbitrary text dᵢ.

$$\begin{matrix}[ Equation 1 ] & \\ p ( {w}_{j} \vert {d}_{i} ) = \frac{exp ( {\vec{w}}_{j} \cdot {\vec{d}}_{i} )}{{Σ}_{k = 1}^{n} exp ( {\vec{w}}_{k} \cdot {\vec{d}}_{i} )} & ( 1 )\end{matrix}$$

Note that the probability P(wⱼ|dᵢ) is a value that can be computed in accordance with a probability p disclosed in, for example, a follow thesis describing evaluation of a text or a document by a paragraph vector. “‘Distributed Representations of Sentences and Documents’ by Quoc Le and Tomas Mikolov, Google Inc; Proceedings of the 31st International Conference on Machine Learning Held in Bejing, China on 22-24 Jun. 2014” This thesis states that, for example, when there are three words “the”, “cat”, and “sat”, “on” is predicted as a fourth word, and a computation formula of the prediction probability p is described. The probability p(wt|wt−k, . . . , wt+k) described in the thesis is a correct answer probability when another word wt is predicted from a plurality of words wt−k, . . . , wt+k.

Meanwhile, the probability P(wⱼ|dᵢ) shown in Equation (1) used in the present embodiment represents a correct answer probability that one word w of n words is predicted from one text dᵢ of m texts. Predicting one word wⱼ from one text dᵢ means that, specifically, when a certain text dᵢ appears, a possibility of including the word wⱼ in the text dᵢ is predicted.

In Equation (1), an exponential function value is used, where e is the base and the inner product of the word vector w→ and the text vector d→ is the exponent. Then, a ratio of an exponential function value calculated from a combination of a text dᵢ and a word wⱼ to be predicted to the sum of n exponential function values calculated from each combination of the text dᵢ and n words wₖ (k=1, 2, . . . , n) is calculated as a correct answer probability that one word wⱼ is expected from one text dᵢ.

Here, the inner product value of the word vector wⱼ→ and the text vector dᵢ→ can be regarded as a scalar value when the word vector wⱼ→ is projected in a direction of the text vector dᵢ→, that is, a component value in the direction of the text vector dᵢ→ included in the word vector wⱼ→, which can be considered to represent a degree at which the word wⱼ contributes to the text dᵢ. Therefore, obtaining the ratio of the exponential function value calculated for one word Wⱼ to the sum of the exponential function values calculated for n words wₖ (k=1, 2, . . . , n) using the exponential function value calculated using the inner product corresponds to obtaining the correct answer probability that one word wⱼ of n words is predicted from one text dᵢ.

Note that since Equation (1) is symmetrical with respect to dᵢ and wⱼ, a probability P(dᵢ|wⱼ) that one text dᵢ of m texts is predicted from one word wⱼ of n words may be calculated. Predicting one text dᵢ from one word wⱼ means that, when a certain word wⱼ appears, a possibility of including the word wⱼ in the text dᵢ is predicted. In this case, an inner product value of the text vector dᵢ→ and the word vector wⱼ→ can be regarded as a scalar value when the text vector dᵢ→ is projected in a direction of the word vector wⱼ→, that is, a component value in the direction of the word vector wⱼ→ included in the text vector dᵢ→, which can be considered to represent a degree at which the text dᵢ contributes to the word wⱼ.

Note that here, a calculation example using the exponential function value using the inner product value of the word vector w→ and the text vector d→ as an exponent has been described. However, the exponential function value may not be used. Any calculation formula using the inner product value of the word vector w→ and the text vector d→ may be used. For example, the probability may be obtained from the ratio of the inner product values itself (however, including performing a predetermined operation for obtaining a positive value as the inner product value at all times (for example, inner product value+1)).

Next, the vector computation unit 12A computes the text vector dᵢ→ and the word vector wⱼ→ that maximize a value L of the sum of the probability P(wⱼ|dᵢ) computed by Equation (1) for all the set S as shown in the following Equation (2). That is, the text vector computation unit 121 and the word vector computation unit 122 compute the probability P(wⱼ|dᵢ) computed by Equation (1) for all combinations of the m texts and the n words, and compute the text vector dᵢ→ and the word vector wⱼ→ that maximize a target variable L using the sum thereof as the target variable L.

$$\begin{matrix}[ Equation 2 ] & \\ L = {Σ}_{d ∈ D} {Σ}_{w ∈ W} £ ( w , d ) p ( w \vert d ) & ( 2 )\end{matrix}$$

Maximizing the total value L of the probability P(wⱼ|dᵢ) computed for all the combinations of the m texts and the n words corresponds to maximizing the correct answer probability that a certain word wⱼ (j=1, 2, . . . , n) is predicted from a certain text dᵢ (i=1, 2, . . . , m). That is, the vector computation unit 12A can be considered to compute the text vector dᵢ→ and the word vector wⱼ→ that maximize the correct answer probability.

Here, in the present embodiment, as described above, the vector computation unit 12A converts each of the m texts dᵢ into a q-dimensional vector to compute the m texts vectors dᵢ→ including the q axis components, and converts each of the n words into a q-dimensional vector to compute the n word vectors wⱼ→ including the q axis components, which corresponds to computing the text vector dᵢ→ and the word vector wⱼ→ that maximize the target variable L by making q axis directions variable.

The index value computation unit 13A takes each of the inner products of the m text vectors dᵢ→ and the n word vectors wⱼ→ computed by the vector computation unit 12A, thereby computing m×n relationship index values reflecting the relationship between them texts dᵢ and the n words wⱼ. In the present embodiment, as shown in the following Equation (3), the index value computation unit 13A obtains the product of a text matrix D having the respective q axis components (d₁₁ to d_{mq}) of the m text vectors dᵢ→ as respective elements and a word matrix W having the respective q axis components (w₁₁ to w_{nq}) of the n word vectors wⱼ→ as respective elements, thereby computing an index value matrix DW having m×n relationship index values as elements. Here, W^{t} is the transposed matrix of the word matrix.

$$\begin{matrix}[ Equation 3 ] & \\ \begin{matrix}D = ( \begin{matrix}{d}_{11} & {d}_{12} & … & {d}_{1 q} \\ {d}_{21} & {d}_{22} & … & {d}_{2 q} \\ ⋮ & ⋮ & ⋱ & ⋮ \\ {d}_{m 1} & {d}_{m 2} & … & {d}_{mq}\end{matrix} ) & W = ( \begin{matrix}{w}_{11} & {w}_{12} & … & {w}_{1 q} \\ {w}_{21} & {w}_{22} & … & {w}_{2 q} \\ ⋮ & ⋮ & ⋱ & ⋮ \\ {w}_{n 1} & {w}_{m 2} & … & {w}_{mq}\end{matrix} )\end{matrix} \\ DW = D * {W}^{t} = ( \begin{matrix}{dw}_{11} & {dw}_{12} & … & {dw}_{1 n} \\ {dw}_{21} & {dw}_{22} & … & {dw}_{2 n} \\ ⋮ & ⋮ & ⋱ & ⋮ \\ {dw}_{m 1} & {dw}_{m 2} & … & {dw}_{mn}\end{matrix} ) & ( 3 )\end{matrix}$$

Each element dwᵢⱼ (i=1, 2, . . . , m; j=1, 2, . . . , n) of the index value matrix DW computed in this manner may indicate which word contributes to which text and to what extent. For example, an element dw₁₂ in the first row and the second column is a value indicating a degree at which the word w₂ contributes to a text d₁. In this way, each row of the index value matrix DW can be used to evaluate the similarity of a text, and each column can be used to evaluate the similarity of a word.

The prediction model generation unit 14A generates a prediction model for predicting severity of dementia based on a text index value group including n relationship index values dwᵢⱼ (j=1, 2, . . . , n) for one text dᵢ using m×n relationship index values computed by the index value computation unit 13A. Here, the severity of the dementia to be predicted is a value of an MMSE score. That is, the prediction model generation unit 14A generates a prediction model in which a score as close to x points as possible is predicted for a text index value group computed based on a free conversation of a patient whose MMSE score is known (for example, x points). Then, the prediction model generation unit 14A causes the prediction model storage unit 30A to store the generated prediction model.

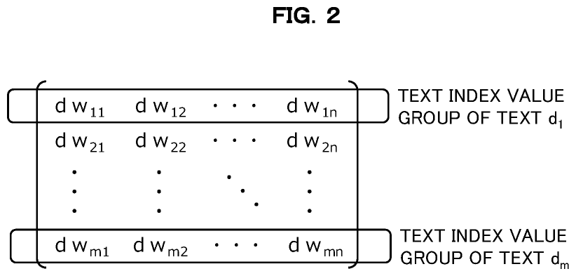

FIG. 2 is a diagram for description of a text index value group. As illustrated in FIG. 2, for example, in the case of the first text d₁, n relationship index values dw₁₁ to dw₁ₙ included in a first row of an index value matrix DW correspond to a text index value group. Similarly, in the case of the second text d₂, n relationship index values dw₂₁ to dw₂ₙ included in a second row of the index value matrix DW correspond to a text index value group. Hereinafter, this description is similarly applied up to a text index value group related to an mth text dₘ (n relationship index values dwₘ₁ to dwₘₙ).

The prediction model generation unit 14A computes each feature quantity associated with severity of dementia for a text index value group of each of the texts dᵢ (i=1, 2, . . . , m) using m×n relationship index values dw₁₁ to dwₘₙ computed by the index value computation unit 13A, and generates a prediction model for predicting severity of dementia from one text index value group based on the computed feature quantity. Here, the prediction model generated by the prediction model generation unit 14A is a learning model in which a text index value group of a text dᵢ is input and an MMSE score is output as a solution.

For example, a form of the prediction model generated by the prediction model generation unit 14A may be set to any one of a regression model (learning model based on linear regression, logistic regression, support vector machine, etc.), a tree model (learning model based on decision tree, regression tree, random forest, gradient boosting tree, etc.), a neural network model (learning model based on perceptron, convolutional neural network, recurrent neural network, residual network, RBF network, stochastic neural network, spiking neural network, complex neural network, etc.), a Bayesian model (learning model based on Bayesian inference), a clustering model (learning model based on k-nearest neighbor method, hierarchical clustering, non-hierarchical clustering, topic model, etc.), etc. Note that the prediction models listed here are merely examples, and the invention is not limited thereto.

The feature quantity computed when the prediction model generation unit 14A generates the prediction model may be computed by a predetermined algorithm. In other words, a method of computing the feature quantity performed by the prediction model generation unit 14A may be arbitrarily designed. For example, the prediction model generation unit 14A performs predetermined weighting calculation on each text index value group of each text dᵢ so that a value obtained by the weighting calculation approaches a known value (MMSE score) indicating the severity of dementia, and generates a prediction model for predicting the severity of dementia (MMSE score) from the text index value group of the text dᵢ using a weighted value for the text index value group as the feature quantity.

In more detail, with regard to the text index value group of the first text d₁ including n relationship index values dw₁₁ to dw₁ₙ contained in the first row of the index value matrix DW, a weighted value {a₁₁, a₁₂, . . . , a₁ₙ} is computed as a feature quantity so that the following expression is satisfied.

a₁₁·dw₁₁+a₁₂·dw₁₂+ . . . a₁ₙ·dw₁ₙ≈known score for MMSE

In addition, with regard to the text index value group of the second text d₂ including n relationship index values dw₂₁ to dw₂ₙ contained in the second row of the index value matrix DW, a weighted value {a₂₁, a₂₂, . . . , a₂ₙ} is computed as a feature quantity so that the following expression is satisfied.

a₂₁·dw₂₁+a₂₂·dw₂₂+ . . . a₂ₙ·dw₂ₙ known score for MMSE

Similarly, with regard to the text index value group of the mth text dₘ, a weighted value {aₘ₁, aₘ₂, . . . , aₘₙ} is computed as a feature quantity so that the following expression is satisfied.

aₘ₁·dwₘ₁+aₘ₂·dwₘ₂+ . . . aₘₙ·dwₘₙ≈known score for MMSE

Then, a prediction model in which each of these feature quantities is associated with a known score for the MMSE is generated.

Note that, here, a description has been given of an example in which each of m sets of weighted values {a₁₁, a₁₂, . . . , a₁ₙ}, . . . , {aₘ₁, aₘ₂, . . . , aₘₙ} are used as feature quantities. However, the invention is not limited thereto. For example, using text index value groups obtained from learning data of patients having the same score for the MMSE among m text index value groups obtained from m pieces of learning data related to the m patients, one or a plurality of weight values having a characteristic common to these text index value or predetermined calculated values using the plurality of weight values, etc. may be extracted as a feature quantity.

The prediction data input unit 20 inputs, as prediction data, m′ texts representing contents of free conversations conducted by m′ patients (m′ is an arbitrary integer of 1 or more) subjected to prediction, respectively. That is, the prediction data input unit 20 replaces voice of a free conversation between a doctor and a patient whose score for the MMSE is unknown with character data, and inputs a text of an utterance part of the patient included in the character data as prediction data. A method of acquiring m′ texts from a free conversation between a doctor and a patient subjected to prediction is similar to the method of acquiring m texts from a free conversation between a doctor and a patient to be learned.

The patient subjected to prediction may be a first-visit patient or a return-visit patient diagnosed with suspected dementia. When the first-visit patient is subjected to prediction, whether or not the patient is suspected of having dementia can be predicted and if the patient has dementia, the severity of the dementia can be predicted only by conducting a free conversation between the patient and the doctor by an interview without performing the MMSE on the patient as described below. Meanwhile, when the return-visit patient is subjected to prediction, the severity of dementia can be predicted only by conducting a free conversation between the patient and the doctor by an interview without performing the MMSE on the patient. In this way, it is possible to determine whether a symptom is ameliorating or worsening without being affected by the practice effect of the patient on the MMSE.

The dementia prediction unit 21A applies a relationship index value obtained by executing processes of the word extraction unit 11A, the text vector computation unit 121, the word vector computation unit 122, and the index value computation unit 13A on prediction data input by the prediction data input unit 20 to a prediction model generated by the prediction model generation unit 14A (prediction model stored in the prediction model storage unit 30A), thereby predicting severity of dementia for m′ patients subjected to prediction.

For example, when m′ texts acquired from free conversations of m′ patients whose scores for the MMSE are unknown are input as prediction data by the prediction data input unit 20, m′ text index value groups are obtained by executing a process of the relationship index value computation unit 100A for the m′ texts according to an instruction of the dementia prediction unit 21A. The dementia prediction unit 21A assigns the m′ text index value groups computed by the relationship index value computation unit 100A to the prediction model as input data, thereby predicting the severity of dementia related to each of the m′ patients.

At the time of this prediction, the word extraction unit 11A extracts n words from the m′ texts input by the prediction data input unit 20 as prediction data. The number of words extracted from the m′ texts by the word extraction unit 11A during prediction is the same as the number n of words extracted from them texts by the word extraction unit 11A during learning. Note that, for example, there is the case of m′=1, that is, a case where n words are extracted from one text by a free conversation of one patient. Therefore, it is preferable to presume a standard type of word spoken by one patient in a free conversation in the form of an interview of about 5 to 10 minutes and determine a value of n so that a situation in which there is no overlap (same word) between n words extracted from one text of prediction data and n words extracted from m texts of learning data does not occur.

In addition, during prediction, the text vector computation unit 121 converts each of m′ texts into a q-dimensional vector according to a predetermined rule, thereby computing m′ text vectors including q axis components. The word vector computation unit 122 converts each of n words into a q-dimensional vector according to a predetermined rule, thereby computing n word vectors including q axis components. The index value computation unit 13A obtains each of inner products of the m′ text vectors and the n word vectors, thereby computing m′×n relationship index values reflecting a relationship between the m′ texts and the n words. The dementia prediction unit 21A applies m′×n relationship index values computed by the index value computation unit 13A to a prediction model stored in the prediction model storage unit 30A, thereby predicting severity of dementia for m′ patients subjected to prediction.

Note that for the purpose of reducing an operation load during prediction, computation of a word vector by the word vector computation unit 122 may be omitted, and n word vectors computed during learning may be stored and used during prediction. In this way, a process of reading and using n word vectors computed during learning by the word vector computation unit 122 during prediction is included as one aspect of executing a process of the word vector computation unit 122 on the prediction data.

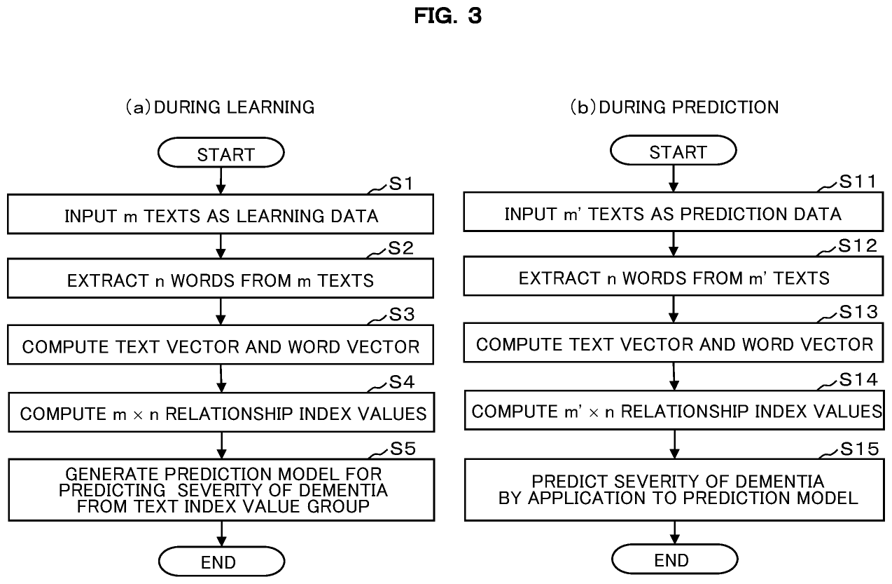

FIG. 3 is a flowchart illustrating an operation example of the dementia prediction device according to the first embodiment configured as described above. FIG. 3(a) illustrates an example of an operation during learning for generating a prediction model, and FIG. 3(b) illustrates an example of an operation during prediction for predicting severity of dementia using the generated prediction model.

During learning illustrated in FIG. 3(a), first, the learning data input unit 10 inputs, as learning data, m texts representing contents of free conversations conducted by m patients whose severity of dementia (score for the MMSE) is known, respectively (step S1). The word extraction unit 11A analyzes the m texts input by the learning data input unit 10, and extracts n words from the m texts (step S2).

Subsequently, the vector computation unit 12A computes m text vectors dᵢ→ and n word vectors wⱼ→ from the m texts input by the learning data input unit 10 and the n words extracted by the word extraction unit 11A (step S3). Then, the index value computation unit 13A obtains each of inner products of the m text vectors dᵢ→ and the n word vectors wⱼ→, thereby computing m×n relationship index values (index value matrix DW having m×n relationship index values as respective elements) reflecting a relationship between the m texts dᵢ and the n words wⱼ (step S4).

Further, as described above, the prediction model generation unit 14A generates a prediction model for predicting severity of dementia based on a text index value group including n relationship index values dwᵢⱼ for one text dᵢ using m×n relationship index values computed by the relationship index value computation unit 100A from learning data related to m patients, and causes the prediction model storage unit 30A to store the generated prediction model (step S5). In this way, an operation during learning is completed.

During prediction illustrated in FIG. 3(b), first, the prediction data input unit 20 inputs m′ texts representing contents of free conversations conducted by m′ patients subjected to prediction, respectively, as prediction data (step S11). The dementia prediction unit 21A supplies the prediction data input by the prediction data input unit 20 to the relationship index value computation unit 100A, and gives an instruction to compute a relationship index value.

In response to this instruction, the word extraction unit 11A analyzes the m′ texts input by the prediction data input unit 20, and extracts n words from the m′ texts (step S12). Subsequently, the vector computation unit 12A computes m′ text vectors dᵢ→ and n word vectors wⱼ→ from the m′ texts input by the prediction data input unit 20 and the n words extracted by the word extraction unit 11A (step S13).

Then, the index value computation unit 13A obtains each of inner products of the m′ text vectors dᵢ→ and the n word vectors wⱼ→, thereby computing m′×n relationship index values (index value matrix DW having m′×n relationship index values as respective elements) reflecting a relationship between the m′ texts dᵢ and the n words wⱼ (step S14). The index value computation unit 13A supplies the computed m′×n relationship index values to the dementia prediction unit 21A.

The dementia prediction unit 21A applies the m′×n relationship index values supplied from the relationship index value computation unit 100A to the prediction model stored in the prediction model storage unit 30A, thereby predicting severity of dementia for m′ patients subjected to prediction (step S15). In this way, an operation during prediction is completed.

As described in detail above, in the first embodiment, m texts representing content of a free conversation conducted by a patient whose severity of dementia is known are input as learning data, an inner product of a text vector computed from the input texts and a word vector computed from words contained in the texts is calculated to compute a relationship index value reflecting a relationship between the texts and the words, and a prediction model is generated using this relationship index value. In addition, when severity of dementia is predicted for a patient subjected to prediction, m′ texts representing content of a free conversation conducted by the patient subjected to prediction are input as prediction data, and a relationship index value similarly computed from the input prediction data is applied to a prediction model, thereby predicting the severity of dementia of the patient subjected to prediction.

According to the first embodiment configured as described above, it is unnecessary to perform a mini-mental state examination (MMSE) since the severity of dementia is predicted by analyzing a free conversation conducted by the patient. Therefore, even when the severity of dementia is repeatedly measured, it is possible to obtain a measurement result (prediction result) excluding the practice effect by the patient. In particular, when a patient suffers from dementia, a conversational characteristic peculiar to dementia, including words repeatedly spoken, can be seen in a free conversation, a relationship index value is computed in a state where such a conversational characteristic is reflected, and a prediction model is generated using the relationship index value. Thus, it is possible to predict the severity of dementia from the free conversation conducted by the patient.

### Second Embodiment

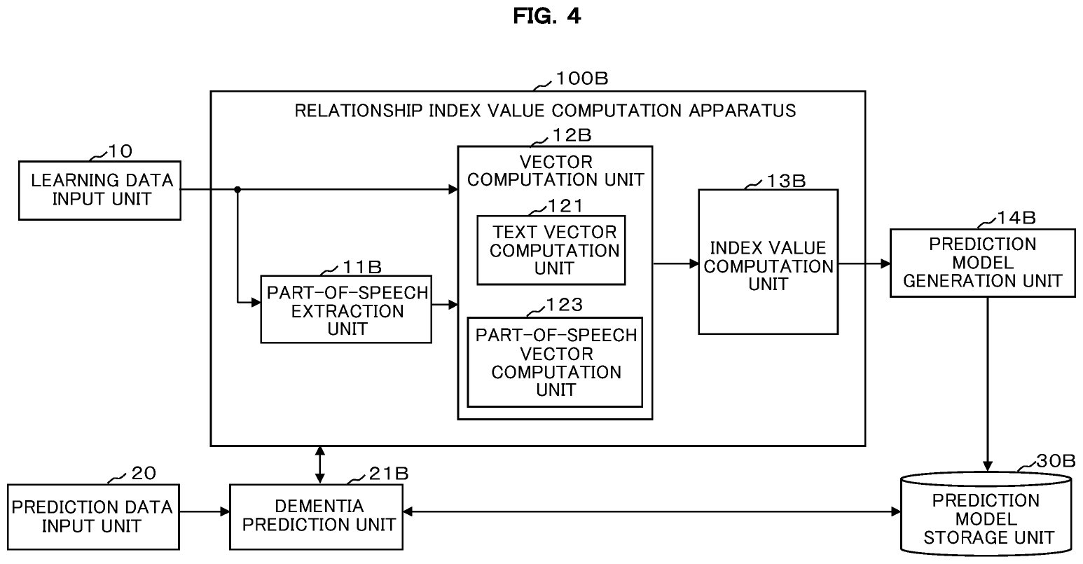

Next, a second embodiment according to the invention will be described with reference to the drawings. FIG. 4 is a block diagram illustrating a functional configuration example of a dementia prediction device according to the second embodiment. In FIG. 4, a component denoted by the same reference symbol as that illustrated in FIG. 1 has the same function, and thus duplicate description will be omitted here.

As illustrated in FIG. 4, the dementia prediction device according to the second embodiment includes a relationship index value computation unit 100B, a prediction model generation unit 14B, a dementia prediction unit 21B, and a prediction model storage unit 30B instead of the relationship index value computation unit 100A, the prediction model generation unit 14A, the dementia prediction unit 21A, and the prediction model storage unit 30A. The relationship index value computation unit 100B according to the second embodiment includes a part-of-speech extraction unit 11B, a vector computation unit 12B, and an index value computation unit 13B instead of the word extraction unit 11A, the vector computation unit 12A, and the index value computation unit 13A. The vector computation unit 12B includes a part-of-speech vector computation unit 123 instead of the word vector computation unit 122 as a more specific functional configuration. Note that a prediction model generation device of the invention includes the learning data input unit 10, the relationship index value computation unit 100B, and the prediction model generation unit 14B.

The relationship index value computation unit 100B according to the second embodiment inputs text data related to a text similar to that of the first embodiment, and computes and outputs a relationship index value reflecting a relationship between the text and a part of speech of each morpheme contained therein.

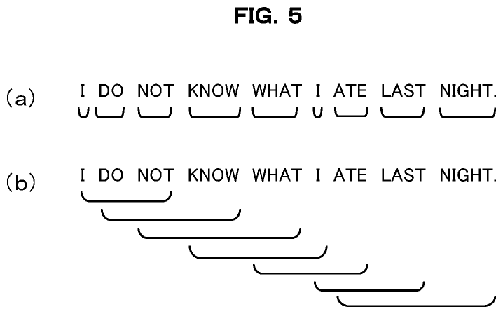

The part-of-speech extraction unit 11B is an example of an “element extraction unit” in the claims, which analyzes m texts input as learning data by the learning data input unit 10, and extracts p parts of speech (p is an arbitrary integer of 2 or more) (corresponding to decomposition elements in the claims) from the m texts. As a text analysis method, for example, it is possible to use a known morphological analysis. Here, for each morpheme divided by the morphological analysis, the part-of-speech extraction unit 11B may extract one part of speech for each single morpheme as illustrated in FIG. 5(a) or extract one set of parts of speech for a plurality of consecutive morphemes as illustrated in FIG. 5(b).

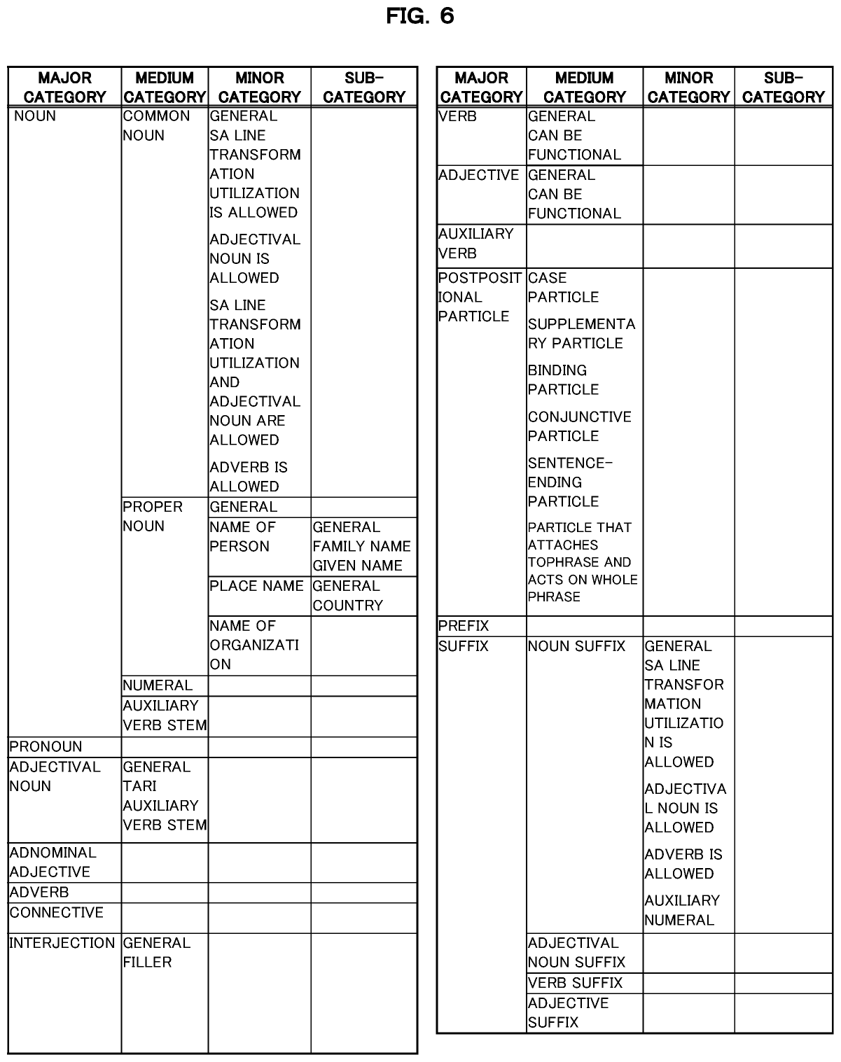

Note that in the present embodiment, as parts of speech to be extracted, parts of speech classified not only into major categories such as a verb, an adjective, an adjective verb, a noun, a pronoun, a numeral, an adnominal adjective, an adverb, a connective, an interjection, an auxiliary verb, and a postpositional particle, but also into a medium category, a minor category, and a sub-category as shown in FIG. 6 are extracted. FIG. 6 shows an example of parts of speech extracted by the part-of-speech extraction unit 11B. The parts of speech illustrated herein are an example, and the invention is not limited thereto.

Note that the same part of speech (or the same set of parts of speech) maybe included in m texts a plurality of times. In this case, the part-of-speech extraction unit 11B does not extract the same part of speech (or the same set of parts of speech) the plurality of times, and extracts the same part of speech (or the same set of parts of speech) only once. In other words, p parts of speech (concept including p sets, which is similarly applied hereinafter) extracted by the part-of-speech extraction unit 11B refers to p types of parts of speech. However, each of the extracted p parts of speech is accompanied by information indicating an appearance frequency in the respective texts.

Patient suffering from dementia may not remember proper nouns and tend to frequently use demonstratives such as “that”, “this”, and “it”. In addition, patients suffering from dementia may tend to frequently use fillers such as “well”, “um”, and “uh” without the following words coming out. For this reason, there is the same part of speech appearing many times in a text of a free conversation according to such a conversational characteristic peculiar to dementia. The part-of-speech extraction unit 11B extracts p parts of speech from the text of the free conversation having such a conversational characteristic peculiar to dementia.

The vector computation unit 12B computes m text vectors and p part-of-speech vectors from m texts and p parts of speech. Here, the text vector computation unit 121 converts each of m texts to be analyzed by the part-of-speech extraction unit 11B into a q-dimensional vector according to a predetermined rule, thereby computing m text vectors including q axis components. In addition, the part-of-speech vector computation unit 123 converts each of p parts of speech extracted by the part-of-speech extraction unit 11B into a q-dimensional vector according to a predetermined rule, thereby computing p part-of-speech vectors including q axis components.

A method of computing the text vectors and the part-of-speech vectors is similar to that of the first embodiment. That is, in the second embodiment, the vector computation unit 12B considers a set S=<d ∈ D, h ∈ H> including m texts and p parts of speech. Here, a text vector dᵢ→ and a part-of-speech vector hⱼ→ are associated with each of texts dᵢ (i=1, 2, . . . , m) and each of parts of speech hⱼ (j=1, 2, . . . , p), respectively. Then, the vector computation unit 12B computes a probability P(hⱼ|dᵢ) computed similarly to the above Equation (1) for all combinations of the m texts and the p parts of speech, sets a total value as a target variable L, and computes a text vector dᵢ→ and a part-of-speech vector hⱼ→ that maximize the target variable L.

The index value computation unit 13B obtains each of inner products of m text vectors dᵢ→ and p part-of-speech vectors hⱼ→ computed by the vector computation unit 12B, thereby computing m×p relationship index values reflecting a relationship between m texts dᵢ and p parts of speech hⱼ. In the second embodiment, as shown in the following Equation (4), the index value computation unit 13B obtains a product of a text matrix D having q respective axis components (d₁₁ to d_{mq}) of the m text vectors dᵢ→ as respective elements and a part-of-speech matrix H having q respective axis components (h₁₁ to h_{pq}) of the p part-of-speech vectors hⱼ→ as respective elements, thereby computing an index value matrix DH having the m×p relationship index values as respective elements. Here, H^{t} is a transposed matrix of the part-of-speech matrix.

$$\begin{matrix}[ Equation 4 ] & \\ D = ( \begin{matrix}{d}_{11} & {d}_{12} & … & {d}_{1 q} \\ {d}_{21} & {d}_{22} & … & {d}_{2 q} \\ ⋮ & ⋮ & ⋱ & ⋮ \\ {d}_{m 1} & {d}_{m 2} & … & {d}_{m q}\end{matrix} ) H = ( \begin{matrix}{h}_{11} & {h}_{12} & … & {h}_{1 q} \\ {h}_{21} & {h}_{22} & … & {h}_{2 q} \\ ⋮ & ⋮ & ⋱ & ⋮ \\ {h}_{p 1} & {h}_{p 2} & … & {d}_{pq}\end{matrix} ) \\ DH = D * {H}^{t} = ( \begin{matrix}{dh}_{11} & {dh}_{12} & … & {dh}_{1 p} \\ {dh}_{21} & {dh}_{22} & … & {dh}_{2 p} \\ ⋮ & ⋮ & ⋱ & ⋮ \\ {dh}_{m 1} & {dh}_{m 2} & … & {dh}_{mp}\end{matrix} ) & ( 4 )\end{matrix}$$

The prediction model generation unit 14B generates a prediction model for predicting severity of dementia (score value for the MMSE) based on a text index value group including p relationship index values dhᵢⱼ (j=1, 2, . . . , p) for one text dᵢ using the m×p relationship index values computed by the index value computation unit 13B. That is, using a similar method to that described in the first embodiment, the prediction model generation unit 14B generates a prediction model in which a score as close to x points as possible is predicted for a text index value group computed based on a free conversation of a patient whose score for the MMSE is known (for example, x points). Then, the prediction model generation unit 14B causes the prediction model storage unit 30B to store the generated prediction model.

The dementia prediction unit 21B applies a relationship index value obtained by executing processes of the part-of-speech extraction unit 11B, the text vector computation unit 121, the part-of-speech vector computation unit 123, and the index value computation unit 13B on prediction data input by the prediction data input unit 20 to a prediction model generated by the prediction model generation unit 14B (prediction model stored in the prediction model storage unit 30B), thereby predicting severity of dementia for m′ patients subjected to prediction.

As described in detail above, in the second embodiment, m texts representing content of a free conversation conducted by a patient whose severity of dementia is known are input as learning data, an inner product of a text vector computed from the input texts and a part-of-speech vector computed from a part of speech of a morpheme contained in the text is calculated, thereby computing a relationship index value reflecting a relationship between the text and the part of speech, and a prediction model is generated using this relationship index value. In addition, when severity of dementia is predicted for a patient subjected to prediction, m′ texts representing content of a free conversation conducted by the patient subjected to prediction are input as prediction data, and a relationship index value similarly computed from the input prediction data is applied to a prediction model, thereby predicting the severity of dementia of the patient subjected to prediction.

Also in the second embodiment configured in this way, since the severity of dementia is predicted by analyzing the free conversation conducted by the patient, it is unnecessary to perform the mini-mental state examination (MMSE). For this reason, even when the severity of dementia is repeatedly measured, it is possible to obtain a measurement result (prediction result) excluding the practice effect by the patient. In particular, when a patient suffers from dementia, a conversational characteristic peculiar to dementia containing a lot of morphemes of a predetermined part of speech can be seen in a free conversation, a relationship index value is computed in a state where such a conversational characteristic is reflected, and a prediction model is generated using the relationship index value. Thus, it is possible to predict the severity of dementia from the free conversation conducted by the patient.

### Third Embodiment

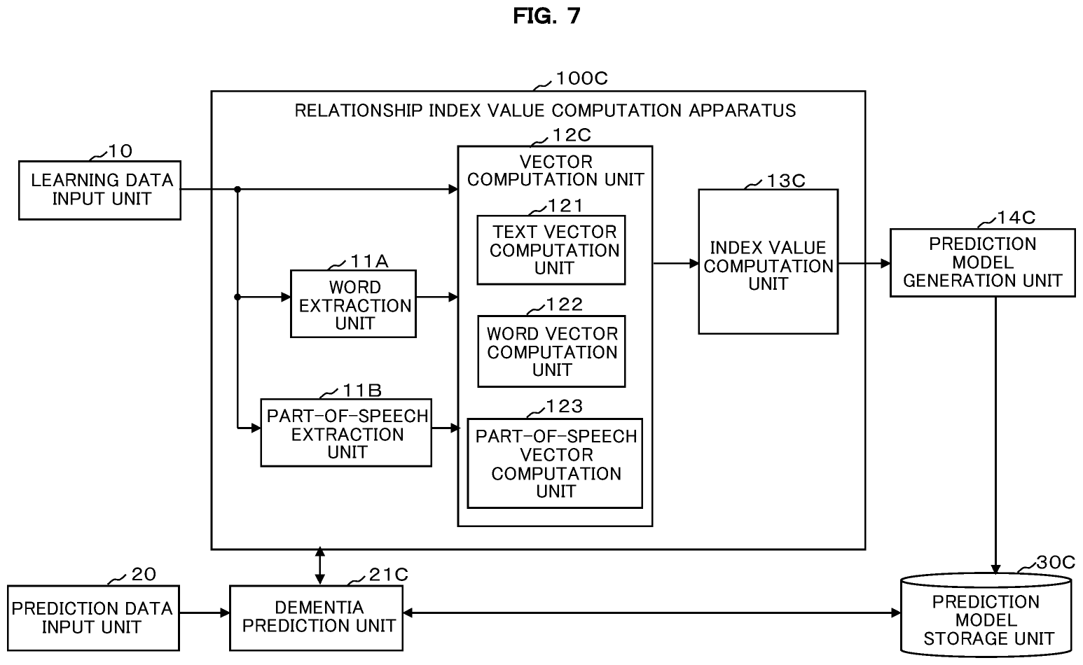

Next, a third embodiment according to the invention will be described with reference to the drawings. FIG. 7 is a block diagram illustrating a functional configuration example of a dementia prediction device according to the third embodiment. In FIG. 7, a component denoted by the same reference symbol as that illustrated in FIG. 4 has the same function, and thus duplicate description will be omitted here. The third embodiment uses both the index value matrix DW computed from the text vector and the word vector described in the first embodiment and the index value matrix DH computed from the text vector and the part-of-speech vector described in the second embodiment.

As illustrated in FIG. 7, the dementia prediction device according to the third embodiment includes a relationship index value computation unit 100C, a prediction model generation unit 14C, a dementia prediction unit 21C, and a prediction model storage unit 30C instead of the relationship index value computation unit 100B, the prediction model generation unit 14B, the dementia prediction unit 21B, and the prediction model storage unit 30B. The relationship index value computation unit 100C according to the third embodiment includes the word extraction unit 11A and the part-of-speech extraction unit 11B, and includes a vector computation unit 12C and an index value computation unit 13C instead of the vector computation unit 12B and the index value computation unit 13B. As a more specific functional configuration, the vector computation unit 12C includes a text vector computation unit 121, a word vector computation unit 122, and a part-of-speech vector computation unit 123. Note that a prediction model generation device of the invention includes the learning data input unit 10, the relationship index value computation unit 100C, and the prediction model generation unit 14C.

As shown in the above Equation (3), the index value computation unit 13C obtains each of inner products of m text vectors dᵢ→ and n word vectors wⱼ→, thereby computing m×n relationship index values dwᵢⱼ (referred to as a first index value matrix DW) reflecting a relationship between m texts dᵢ and n words wⱼ. In addition, as shown in the above Equation (4), the index value computation unit 13C obtains each of inner products of m text vectors dᵢ→ and p part-of-speech vectors hⱼ→, thereby computing m×p relationship index values dhᵢⱼ (referred to as a second index value matrix DH) reflecting a relationship between m texts dᵢ and p parts of speech hⱼ.

The prediction model generation unit 14C generates a prediction model for predicting severity of dementia (score value for the MMSE) based on a text index value group dwᵢⱼ (j=1, 2, . . . , n) including n relationship index values and a text index value group dhᵢⱼ (j=1, 2, . . . , p) including p relationship index values for one text dᵢ using m×n relationship index values dwᵢⱼ and m×p relationship index values dhᵢⱼ computed by the index value computation unit 13C. Then, the prediction model generation unit 14C causes the prediction model storage unit 30C to store the generated prediction model.

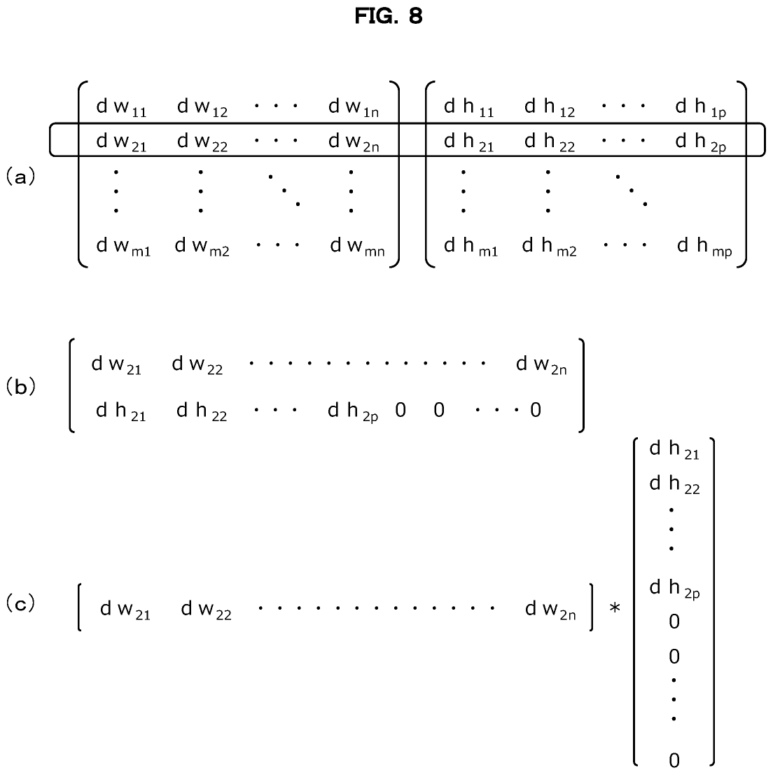

Here, it is possible to arbitrarily design a scheme in which the prediction model generation unit 14C uses two sets of text index value groups dwᵢⱼ and dhᵢⱼ to generate a prediction model. For example, as illustrated in FIG. 8(a), the first index value matrix DW between texts/words and the second index value matrix DH between texts/parts of speech may be arranged horizontally (row direction), text index value groups dwᵢⱼ and dhᵢⱼ belonging to the same row i may be connected to generate one text index value group including (n+p) relationship index values, and a prediction model for predicting severity of dementia may be generated based on this text index value group.

Alternatively, as illustrated in FIG. 8(b), a text index value group dwᵢⱼ on an ith row included in the first index value matrix DW between texts/words and a text index value group dhᵢⱼ on the same ith row included in the second index value matrix DH between texts/parts of speech may be arranged vertically (column direction) to generate a (2×n)-dimensional text index value group matrix, and a prediction model for predicting severity of dementia maybe generated based on this text index value group matrix. In the example of FIG. 8(b), n>p is presumed, values of the text index value group dhᵢⱼ are set left-justified for matrix components of a second row in the (2×n)-dimensional text index value group matrix, and values of all matrix components exceeding p from a left end of the second row are set to 0.

Note that an (m×p) -dimensional first index value matrix DW_{SVD} may be generated by performing dimensional compression processing which will be described later in a fourth embodiment on an (m×n)-dimensional first index value matrix DW, a (2×p)-dimensional text index value group matrix may be generated by vertically (column direction) arranging a text index value group dwᵢⱼ (j=1 to p) on an ith row contained in this dimensionally compressed first index value matrix DW_{SVD} and a text index value group dhᵢⱼ (j=1 to p) on the same ith row contained in the second index value matrix DH, and a prediction model for predicting severity of dementia may be generated based on this text index value group matrix.

As yet another example, as in FIG. 8(c), a text index value group dwᵢⱼ on the ith row contained in the first index value matrix DW between texts/words is set to a (1×n)-dimensional first text index value group matrix, and a text index value group dhᵢⱼ on the same ith row contained in the second index value matrix DH between texts/parts of speech is set to an (n×1)-dimensional second text index value group matrix (however, a value of a matrix component corresponding to a surplus of p and a shortage of n is set to 0), thereby calculating an inner product of the first text index value group matrix and the second text index value group matrix. Then, a prediction model for predicting severity of dementia may be generated based on the calculated value.

In this case, the (m×p)-dimensional first index value matrix DW_{SVD} may be generated by dimensionally compressing the first index value matrix DW between texts/words, and the inner product of the first text index value group matrix and the second text index value group matrix may be calculated by setting the text index value group dwᵢⱼ on the ith row contained in this dimensionally compressed first index value matrix DW_{SVD} to a (1×p)-dimensional first text index value group matrix and setting the text index value group dhᵢⱼ on the same ith row contained in the second index value matrix DH between texts/parts of speech to a (p×1)-dimensional second text index value group matrix.

The dementia prediction unit 21C applies a relationship index value obtained by executing processes of the word extraction unit 11A, the part-of-speech extraction unit 11B, the text vector computation unit 121, the word vector computation unit 122, the part-of-speech vector computation unit 123, and the index value computation unit 13C on prediction data input by the prediction data input unit 20 to a prediction model generated by the prediction model generation unit 14C (prediction model stored in the prediction model storage unit 30C), thereby predicting severity of dementia for m′ patients subjected to prediction.

As described in detail above, in the third embodiment, m texts representing content of a free conversation conducted by a patient whose severity of dementia is known are input as learning data, an inner product of a text vector computed from the input texts and a word vector computed from words contained in the texts is calculated to compute a relationship index value reflecting a relationship between the texts and the words, an inner product of a text vector computed from the input texts and a part-of-speech vector computed from parts of speech of morphemes contained in the texts is calculated to compute a relationship index value reflecting a relationship between the texts and the parts of speech, and a prediction model is generated using these relationship index values. In addition, when severity of dementia is predicted for a patient subjected to prediction, m′ texts representing content of a free conversation conducted by the patient subjected to prediction are input as prediction data, and a relationship index value similarly computed from the input prediction data is applied to a prediction model, thereby predicting severity of dementia of the patient subjected to prediction.

Also in the third embodiment configured in this way, since the severity of dementia is predicted by analyzing the free conversation conducted by the patient, it is unnecessary to perform the mini-mental state examination (MMSE). For this reason, even when the severity of dementia is repeatedly measured, it is possible to obtain a measurement result (prediction result) excluding the practice effect by the patient. In particular, in the third embodiment, since a relationship index value is computed in a state where a conversational characteristic peculiar to dementia is computed for a word and a part of speech used during a free conversation, and a prediction model is generated using the relationship index value, it is possible to more accurately predict the severity of dementia from the free conversation conducted by the patient.

### Fourth Embodiment

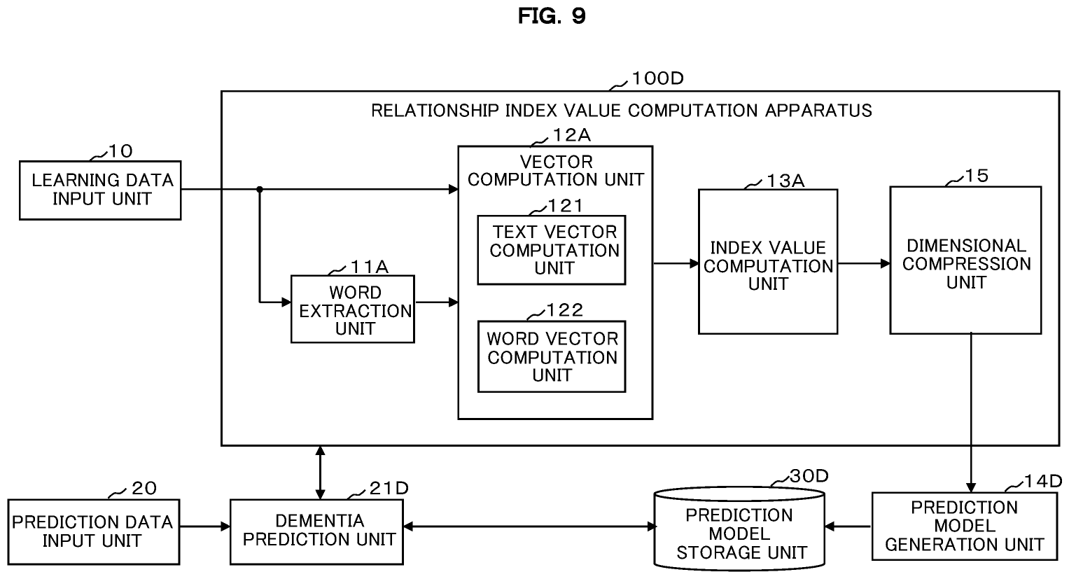

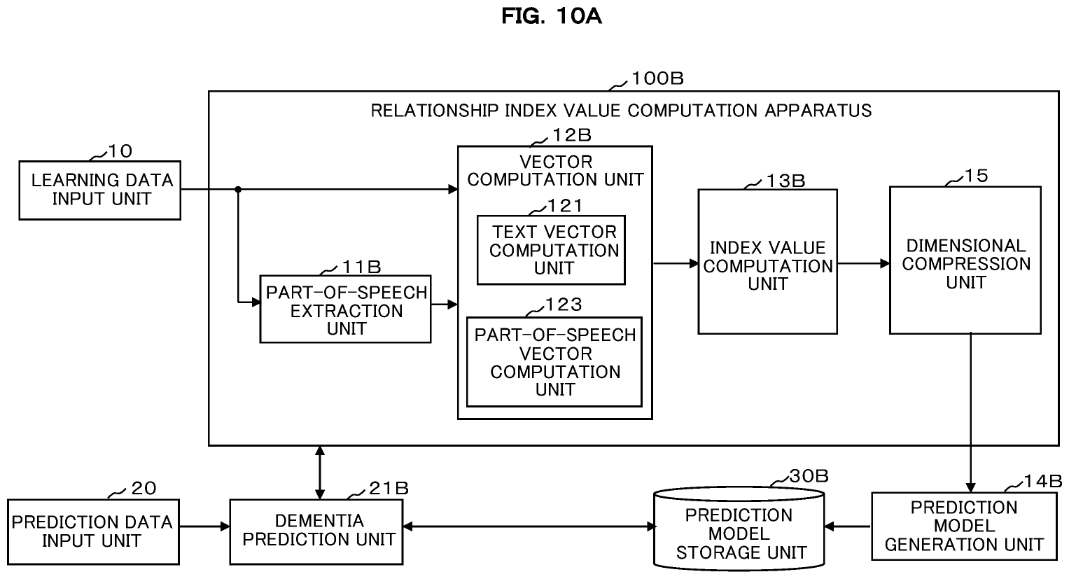

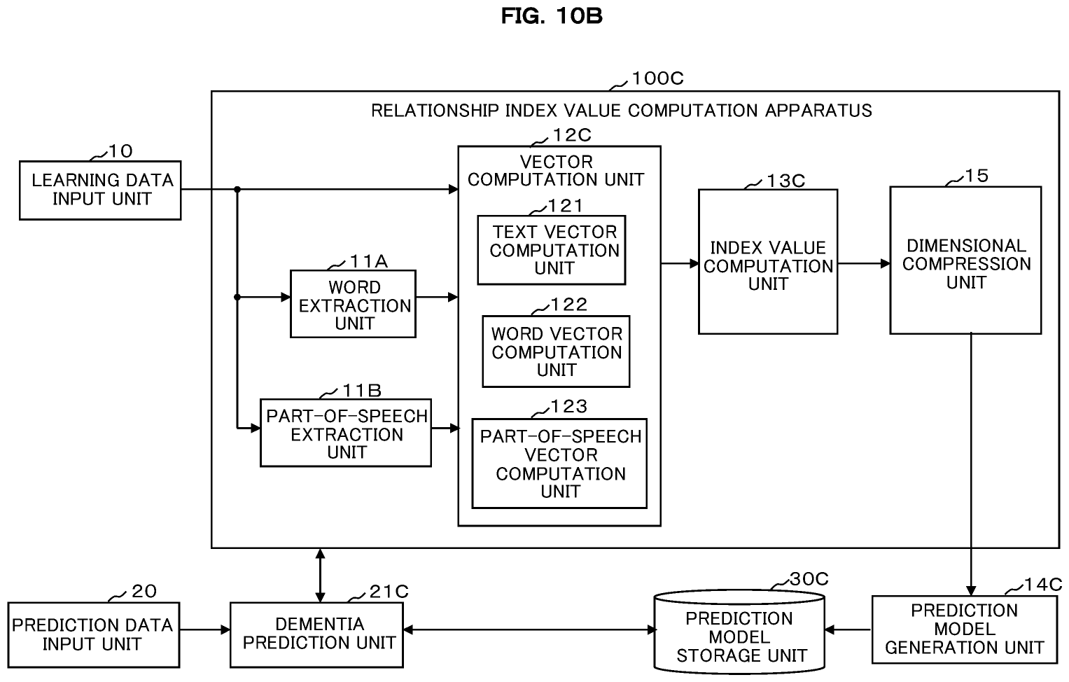

Next, a fourth embodiment according to the invention will be described with reference to the drawings. FIG. 9 is a block diagram illustrating a functional configuration example of a dementia prediction device according to the fourth embodiment. In FIG. 9, a component denoted by the same reference symbol as that illustrated in FIG. 1 has the same function, and thus duplicate description will be omitted here. Note that hereinafter, the fourth embodiment will be described as a modification to the first embodiment. However, as illustrated in each of FIGS. 10A and 10B, the fourth embodiment can be similarly applied as a modification to the second embodiment or a modification to the third embodiment.

As illustrated in FIG. 9, the dementia prediction device according to the fourth embodiment includes a relationship index value computation unit 100D, a prediction model generation unit 14D, a dementia prediction unit 21D, and a prediction model storage unit 30D instead of the relationship index value computation unit 100A, the prediction model generation unit 14A, the dementia prediction unit 21A, and the prediction model storage unit 30A. The relationship index value computation unit 100D according to the fourth embodiment further includes a dimensional compression unit 15 in addition to the configuration illustrated in FIG. 1. Note that a prediction model generation device of the invention includes the learning data input unit 10, the relationship index value computation unit 100D, and the prediction model generation unit 14D.

The dimensional compression unit 15 performs predetermined dimensional compression processing using m×n relationship index values computed by the index value computation unit 13A, thereby computing m×k relationship index values (k is an arbitrary integer satisfying 1≤k<n). In the dimensional compression processing, for example, known singular value decomposition (SVD) maybe used as a method for decomposing a matrix.

That is, the dimensional compression unit 15 decomposes the index value matrix DW computed as in the above Equation (3) into three matrices U, S, and V. Here, the matrix U is an (m×k)-dimensional left singular matrix, in which each column is an eigenvector of DW*DW^{t} (DW^{t} denotes the transposed matrix of the index value matrix DW). The matrix S is a (k×k)-dimensional square matrix, in which a diagonal matrix component indicates a singular value of the index value matrix DW, and all other values are 0. The matrix V is a (k×n)-dimensional right singular matrix, in which each row is an eigenvector of DW^{t}*DW. Note that the dimension k after compression may be a fixed value determined in advance, or an arbitrary value may be specified.

The dimensional compression unit 15 compresses the dimension of the index value matrix DW by transforming the index value matrix DW by the transposed matrix V^{t} of the right singular matrix V among the three matrices decomposed as described above. That is, by calculating an inner product of the (m×n)-dimensional index value matrix DW and the (n×k)-dimensional right singular transposed matrix V^{t}, the (m×n)-dimensional index value matrix DW is dimensionally compressed into the (m×k) -dimensional index value matrix DW_{SVD} (DW_{SVD}=DW*V^{t}). Note that DW_{SVD} denotes a matrix obtained by dimensionally compressing the index value matrix DW using the SVD, and a relationship of DW≈U*S*V=DW_{SVD}*V is established.

By compressing the dimension of the index value matrix DW using the SVD method in this way, the index value matrix DW can be low-rank approximated without impairing a characteristic represented by the index value matrix DW as much as possible. Here, an example of transforming the index value matrix DW by the transposed matrix V^{t} of the right singular matrix V has been described. However, when the value of m is identical to the value of n, the index value matrix DW may be transformed by the left singular matrix U (DW_{SVD}=DW*U).

The prediction model generation unit 14D generates a prediction model for predicting severity of dementia based on a text index value group including k relationship index values dwᵢⱼ (i=1, 2, . . . , k) for one text dᵢ using m×k relationship index values dimensionally compressed by the dimensional compression unit 15. Then, the prediction model generation unit 14D causes the prediction model storage unit 30D to store the generated prediction model.

The dementia prediction unit 21D applies a relationship index value obtained by executing processes of the word extraction unit 11A, the text vector computation unit 121, the word vector computation unit 122, the index value computation unit 13A, and the dimensional compression unit 15 on prediction data input by the prediction data input unit 20 to a prediction model generated by the prediction model generation unit 14D (prediction model stored in the prediction model storage unit 30D), thereby predicting severity of dementia for m′ patients subjected to prediction.

In the first embodiment, it is necessary to select a value of n by presuming a standard type of word spoken by one patient in a free conversation in the form of an interview of about 5 to 10 minutes . When the value of n is small, the word spoken by the one patient subjected to prediction and n types of words extracted from a text of learning data are less overlapped, and there is a possibility that there will be no overlap. In addition, information about a word not included in the n words (word not extracted by the word extraction unit 11) is not added to the index value matrix DW. For this reason, as the value of n decreases, accuracy of prediction decreases. Meanwhile, when a sufficiently large value of n is selected, a possibility that there will be no overlap decreases, and fewer words are not included in the n words. However, a size of the matrix increases, and the amount of calculation increases. In addition, a word having a low appearance frequency is included as a feature quantity, and overfitting is likely to occur.

On the other hand, according to the fourth embodiment, it is possible to extract a lot of (for example, all) words included in m texts as n words to generate an index value matrix DW, and it is possible to compute an index value matrix DW_{SVD} that is dimensionally compressed in a state where a characteristic expressed by this index value matrix DW is reflected. According to this, a prediction model is generated by learning, and severity of dementia is predicted using the generated prediction, more accurately with a small calculation load.

Note that, here, an example using the SVD as an example of dimensional compression has been described. However, the invention is not limited thereto. For example, other dimensional compression methods such as principal component analysis (PCA) may be used.

In addition, in FIG. 9, a description has been given of an example of dimensionally compressing the index value matrix DW between texts/words generated in the first embodiment. However, similar operation can be performed in the case of dimensionally compressing the index value matrix DH between texts/parts of speech generated in the second embodiment as in FIG. 10A. On the other hand, an operation can be performed in the following mode in the case of dimensionally compressing the first index value matrix DW and the second index value matrix DH generated in the third embodiment as in FIG. 10B.

For example, it is possible to individually perform dimensional compression on each of the first index value matrix DW and the second index value matrix DH. That is, an (m×n)-dimensional first index value matrix DW is dimensionally compressed into an (m×k)-dimensional first index value matrix DW_{SVD}, and an (m×p)-dimensional second index value matrix DH is dimensionally compressed into an (m×k)-dimensional second index value matrix DH_{SVD}. As another example, as illustrated in FIG. 8(a), the first index value matrix DW and the second index value matrix DH maybe horizontally arranged to generate one m×(n+p)-dimensional index value matrix, and this generated index value matrix may be dimensionally compressed into an (m×k)-dimensional index value matrix.

### Fifth Embodiment

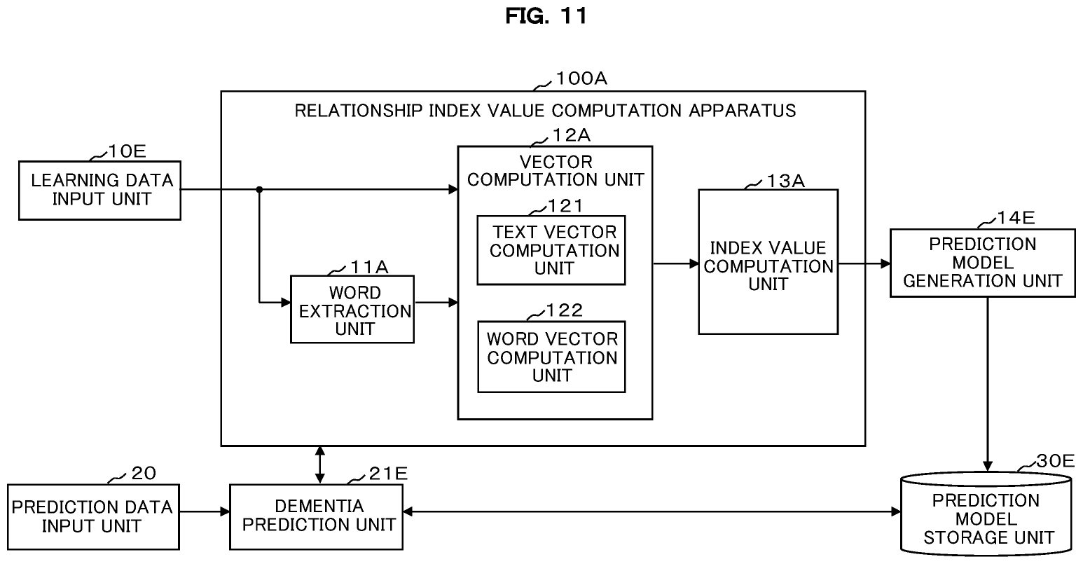

Next, a fifth embodiment according to the invention will be described with reference to the drawings. FIG. 11 is a block diagram illustrating a functional configuration example of a dementia prediction device according to the fifth embodiment. In FIG. 11, a component denoted by the same reference symbol as that illustrated in FIG. 1 has the same function, and thus duplicate description will be omitted here. Note that hereinafter, the fifth embodiment will be described as a modification to the first embodiment. However, the fifth embodiment can be similarly applied as a modification to any one of the second embodiment to the fourth embodiment.

As illustrated in FIG. 11, the dementia prediction device according to the fifth embodiment includes a learning data input unit 10E, a prediction model generation unit 14E, a dementia prediction unit 21E, and a prediction model storage unit 30E instead of the learning data input unit 10, the prediction model generation unit 14A, the dementia prediction unit 21A, and the prediction model storage unit 30A. Note that a prediction model generation device of the invention includes the learning data input unit 10E, the relationship index value computation unit 100A, and the prediction model generation unit 14E.

The learning data input unit 10E inputs, as learning data, m texts representing contents of free conversations conducted by m patients whose severity is known, respectively, for each of a plurality of evaluation items of dementia. The severity of the dementia for each of the plurality of evaluation items means a value of each score of each of five evaluation items of the MMSE, that is, each of insight, memory, attention (calculation), linguistic ability, and composition ability (graphical ability).

The prediction model generation unit 14E generates a prediction model for predicting severity of dementia for each evaluation item based on a text index value group including n relationship index values dwᵢⱼ (j=1, 2, . . . , n) for one text dᵢ using m×n relationship index values computed by the relationship index value computation unit 100A. Here, the severity of dementia to be predicted is a value of a score of each of five evaluation items of the MMSE.

That is, the prediction model generation unit 14E generates a prediction model in which a score as close as possible to ×1 point, ×2 points, ×3 points, ×4 points, and ×5 points is predicted for each evaluation item for a text index value group computed based on a free conversation of a patient whose score of each of insight, memory, attention, linguistic ability, and composition ability of the MMSE (for example, ×1 point, ×2 points, ×3 points, ×4 points, and ×5 points, respectively) is known. Then, the prediction model generation unit 14E causes the prediction model storage unit 30E to store the generated prediction model.

The prediction model generation unit 14E computes a feature quantity associated with severity of dementia for each evaluation item for each text index value group of each text dᵢ (i=1, 2, . . . , m) using m×n relationship index values dw₁₁ to dwₘₙ computed by the index value computation unit 13A for each evaluation item, and generates a prediction model for predicting severity of dementia for each evaluation item from one text index value group based on the computed feature quantity. Here, the prediction model generated by the prediction model generation unit 14E is a learning model in which a text index value group of a text dᵢ is input and a score for each evaluation item of the MMSE is output as a solution.

Also in the fifth embodiment, the feature quantity computed when the prediction model generation unit 14E generates the prediction model may be computed by a predetermined algorithm. In other words, a method of computing the feature quantity performed by the prediction model generation unit 14E can be arbitrarily designed. For example, the prediction model generation unit 14E performs predetermined weighting calculation on each text index value group of each text dᵢ for each evaluation item so that a value obtained by weighting calculation approaches a known value representing the severity of the dementia for each evaluation item (score for each evaluation item of the MMSE), and generates a prediction model for predicting the severity of dementia for each evaluation item (score for each evaluation item of the MMSE) from the text index value group of the text dᵢ using a weighted value for the text index value group as the feature quantity for each evaluation item.

For example, the prediction model generation unit 14E generates a prediction model in which a score of a first evaluation item (insight) is predicted using one or more weight values among n weight values {aᵢ₁, aᵢ₂, . . . , aᵢₙ} for a text index value group of a text dᵢ as a feature quantity, a score of a second evaluation item (memory) is predicted using another one or more weight values as a feature quantity, and similarly, scores of a third evaluation item to a fifth evaluation item (attention, linguistic ability, and composition ability) are predicted using yet another one or more weight values as a feature quantity.

The dementia prediction unit 21E applies a relationship index value obtained by executing processes of the word extraction unit 11A, the text vector computation unit 121, the word vector computation unit 122, and the index value computation unit 13A on prediction data input by the prediction data input unit 20 to a prediction model generated by the prediction model generation unit 14E (prediction model stored in the prediction model storage unit 30E), thereby predicting severity of dementia for each evaluation item for m′ patients subjected to prediction.

According to the fifth embodiment configured as described above, it is possible to predict the score for each evaluation item of the MMSE without performing the mini-mental state examination (MMSE).

Note that even though a description has been given of an example of predicting the score for each of the five evaluation items of the MMSE here, the score may be predicted for each of more evaluation items obtained by further subdividing the five evaluation items.

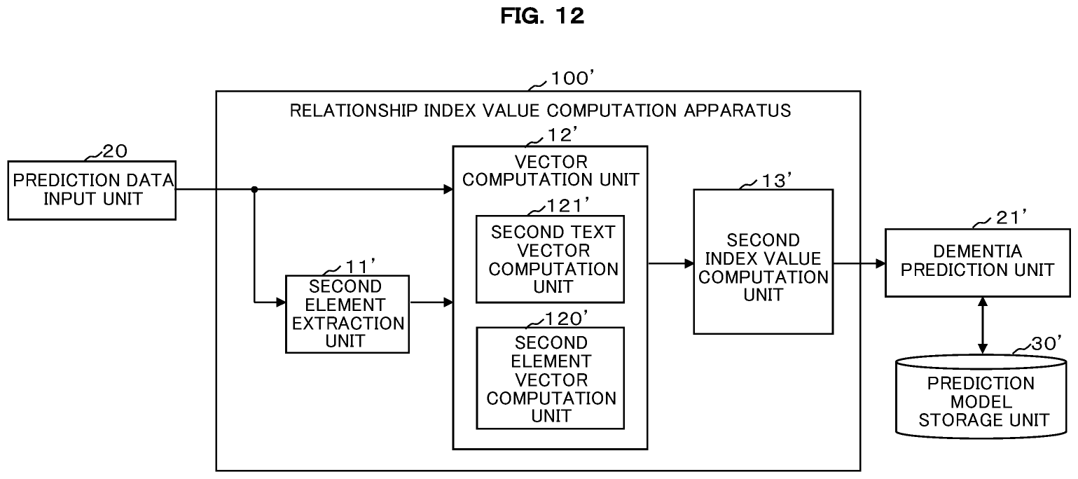

In the first to fifth embodiments, the dementia prediction device including a learner and a predictor has been illustrated. However, it is possible to separately configure a prediction model generation device having only the learner and a dementia prediction device having only the predictor. A configuration of the prediction model generation device including only the learner is as described in the first to fifth embodiments. On the other hand, for example, a configuration of the dementia prediction device including only the predictor is as illustrated in FIG. 12.

In FIG. 12, a second element extraction unit 11′ has a similar function to that of any of the word extraction unit 11A, the part-of-speech extraction unit 11B, or a combination of the word extraction unit 11A and the part-of-speech extraction unit 11B. A second text vector computation unit 121′ has a similar function to that of the text vector computation unit 121. A second element vector computation unit 120′ has a similar function to that any of the word vector computation unit 122, the part-of-speech vector computation unit 123, or a combination of the word vector computation unit 122 and the part-of-speech vector computation unit 123. A second index value computation unit 13′ has a similar function to that of any of the index value computation units 13A to 13E. A dementia prediction unit 21′ has a similar function to that of any of the dementia prediction units 21A to 21E. A prediction model storage unit 30′ stores a prediction model similar to that of any of the prediction model storage units 30A to 30E.

Further, in the first to fifth embodiments, a description has been given of an example of a case where the “severity of dementia” is the score for the MMSE, that is, an example of predicting the score for the MMSE. However, the invention is not limited thereto. For example, the severity of dementia may be set to categories classified by the number less than a maximum value of the score for the MMSE and greater than or equal to 2. For example, the severity of dementia may be classified into three categories such that there is no suspicion of dementia when the MMSE score is 30 to 27 points, mild dementia disorder is suspected when the MMSE score is 26 to 22 points, and dementia is suspected when the MMSE score is 21 points or less, and a category to which the patient corresponds may be predicted.

In this case, for example, in the first embodiment, the prediction model generation unit 14A generates a prediction model in which a text index value group computed based on text data corresponding to a free conversation of a patient whose MMSE score is known to be 30 to 27 points is classified into a first category of “there is no suspicion of dementia”, a text index value group computed based on text data corresponding to a free conversation of a patient whose MMSE score is known to be 26 to 22 points is classified into a second category of “mild dementia disorder is suspected”, and a text index value group computed based on text data corresponding to a free conversation of a patient whose MMSE score is known to be less than 21 points is classified into a third category of “dementia is suspected”.

For example, the prediction model generation unit 14A computes each feature quantity for a text index value group of each text dᵢ, and optimizes categorization by the Markov chain Monte Carlo method according to a value of the computed feature quantity, thereby generating a prediction model for classifying each text dᵢ into a plurality of categories. Here, the prediction model generated by the prediction model generation unit 14A is a learning model that inputs a text index value group and outputs any one of a plurality of categories to be predicted as a solution. Alternatively, the prediction model may be a learning model that outputs a probability of being classified into any category as a numerical value. The form of the learning model is arbitrary.

Further, in the first to fifth embodiments, a description has been given of an example of predicting the severity of dementia based on the MMSE score. However, the invention is not limited thereto. That is, it is possible to predict the severity of dementia based on a method of detecting the severity of dementia other than the MMSE score, for example, the revised Hasegawa's Dementia Scale-Revised (HDS-R), Alzheimer's Disease Assessment Scale-cognitive subscale (ADAS-cog), Clinical Dementia Rating (CDR), Clock Drawing Test (CDT), Neurobehavioral Cognitive Status Examination (COGNISTAT), Seven Minutes Screening, etc.

Further, in the first to fifth embodiments, a description has been given of an example in which a free conversation between a doctor and a patient in the form of an interview is converted into character data and used for learning and prediction related to the severity of dementia. However, the invention is not limited thereto. For example, a free conversation conducted by a patient in daily life may be converted into character data and used for learning and prediction related to the severity of dementia.

In addition, the first to fifth embodiments are merely examples of a specific embodiment for carrying out the invention, and the technical scope of the invention should not be interpreted in a limited manner. That is, the invention can be implemented in various forms without departing from the gist or the main features thereof.

### REFERENCE SIGNS LIST

10, 10E Learning data input unit

11A Word extraction unit (element extraction unit)

11B Part-of-speech extraction unit (element extraction unit)

12A to 12E Vector computation unit

121 Text vector computation unit (element vector computation unit)

122 Word vector computation unit (element vector computation unit)

123 Part-of-speech vector computation unit (element vector computation unit)

13A to 13C Index value computation unit

14A to 14E Prediction model generation unit

15 Dimensional compression unit

20 Prediction data input unit

21A to 21E Dementia prediction unit

30A to 30E Prediction model storage unit

100A to 100E Relationship index value computation unit

1. A dementia prediction device characterized by comprising:
a learning data input unit that inputs a plurality of texts representing contents of free conversations conducted by a plurality of patients whose severity of dementia is known, respectively, as learning data;
an element extraction unit that analyzes morphemes of the plurality of texts input by the learning data input unit as the learning data, and extracts a plurality of decomposition elements from the plurality of texts;
a text vector computation unit that converts each of the plurality of texts into a q- dimensional vector (q is an arbitrary integer of 2 or more) according to a predetermined rule, thereby computing a plurality of text vectors including q axis components;
an element vector computation unit that converts each of the plurality of decomposition elements into a q-dimensional vector according to a predetermined rule, thereby computing a plurality of element vectors including q axis components;
an index value computation unit that obtains each of inner products of the plurality of text vectors and the plurality of element vectors, thereby computing a relationship index value reflecting a relationship between the plurality of texts and the plurality of decomposition elements;
a prediction model generation unit that generates a prediction model for predicting the severity of the dementia based on a text index value group including a plurality of relationship index values for one text using the relationship index value computed by the index value computation unit;
a prediction data input unit that inputs a text representing content of a free conversation conducted by a patient subjected to prediction as prediction data; and
a dementia prediction unit that predicts the severity of the dementia for the patient subjected to prediction by applying a relationship index value obtained by executing processes of the element extraction unit, the text vector computation unit, the element vector computation unit, and the index value computation unit on the prediction data input by the prediction data input unit to the prediction model generated by the prediction model generation unit.

2. The dementia prediction device according to claim 1,
characterized in that the learning data input unit inputs, as the learning data, m texts representing contents of free conversations conducted by m patients (m is an arbitrary integer of 2 or more) whose severity of dementia is known, respectively,
the element extraction unit is a word extraction unit that analyzes the m texts input as the learning data by the learning data input unit and extracts n words (n is an arbitrary integer of 2 or more) from the m texts,
the text vector computation unit converts each of the m texts into a q-dimensional vector according to a predetermined rule, thereby computing m text vectors including q axis components,
the element vector computation unit is a word vector computation unit that converts each of the n words into a q-dimensional vector according to a predetermined rule, thereby computing n word vectors including q axis components,
the index value computation unit obtains each of inner products of the m text vectors and the n word vectors, thereby computing m □ n relationship index values reflecting a relationship between the m texts and the n words,
the prediction model generation unit generates a prediction model for predicting the severity of the dementia based on a text index value group including n relationship index values for one text using the m □ n relationship index values computed by the index value computation unit,
the prediction data input unit inputs, as prediction data, m′ texts representing contents of free conversations conducted by m′ patients (m′ is an arbitrary integer of 1 or more) subjected to prediction, respectively, and
the dementia prediction unit predicts the severity of the dementia for the m′ patients subjected to prediction by applying a relationship index value obtained by executing processes of the word extraction unit, the text vector computation unit, the word vector computation unit, and the index value computation unit on the prediction data input by the prediction data input unit to the prediction model generated by the prediction model generation unit.

3. The dementia prediction device according to claim 1,
characterized in that the learning data input unit inputs, as the learning data, m texts representing contents of free conversations conducted by m patients (m is an arbitrary integer of 2 or more) whose severity of dementia is known, respectively,
the element extraction unit is a part-of-speech extraction unit that analyzes the m texts input as the learning data by the learning data input unit and extracts p parts of speech (p is an arbitrary integer of 2 or more) from the m texts,
the text vector computation unit converts each of the m texts into a q-dimensional vector according to a predetermined rule, thereby computing m text vectors including q axis components,
the element vector computation unit is a part-of-speech vector computation unit that converts the p parts of speech into a q-dimensional vector according to a predetermined rule, thereby computing p part-of-speech vectors including q axis components,
the index value computation unit obtains each of inner products of the m text vectors and the p part-of-speech vectors, thereby computing m □ p relationship index values reflecting a relationship between the m texts and the p parts of speech,
the prediction model generation unit generates a prediction model for predicting the severity of the dementia based on a text index value group including p relationship index values for one text using the m □ p relationship index values computed by the index value computation unit, and
the dementia prediction unit predicts the severity of the dementia for the m′ patients subjected to prediction by applying a relationship index value obtained by executing processes of the part-of-speech extraction unit, the text vector computation unit, the part-of-speech vector computation unit, and the index value computation unit on the prediction data input by the prediction data input unit to the prediction model generated by the prediction model generation unit.

4. The dementia prediction device according to claim 1,
characterized in that the learning data input unit inputs, as the learning data, m texts representing contents of free conversations conducted by m patients (m is an arbitrary integer of 2 or more) whose severity of dementia is known, respectively,
the element extraction unit includes a word extraction unit that analyzes the m texts input as the learning data by the learning data input unit and extracts n words (n is an arbitrary integer of 2 or more) from the m texts, and a part-of-speech extraction unit that analyzes the m texts input as the learning data by the learning data input unit and extracts p parts of speech (p is an arbitrary integer of 2 or more) from the m texts,
the text vector computation unit converts each of the m texts into a q-dimensional vector according to a predetermined rule, thereby computing m text vectors including q axis components,
the element vector computation unit includes a word vector computation unit that converts each of the n words into a q-dimensional vector according to a predetermined rule, thereby computing n word vectors including q axis components, and a part-of-speech vector computation unit that converts each of the p parts of speech into a q-dimensional vector according to a predetermined rule, thereby computing p part-of-speech vectors including q axis components,
the index value computation unit obtains each of inner products of the m text vectors and the n word vectors, thereby computing m □ n relationship index values reflecting a relationship between the m texts and the n words, and obtains each of inner products of the m text vectors and the p part-of-speech vectors, thereby computing m □ p relationship index values reflecting a relationship between the m texts and the p parts of speech,
the prediction model generation unit generates a prediction model for predicting the severity of the dementia based on a text index value group including n relationship index values and a text index value group including p relationship index values for one text using the m □ n relationship index values and the m □ p relationship index values computed by the index value computation unit, and
the dementia prediction unit predicts the severity of the dementia for the m′ patients subjected to prediction by applying a relationship index value obtained by executing processes of the word extraction unit, the part-of-speech extraction unit, the text vector computation unit, the word vector computation unit, the part-of-speech vector computation unit, and the index value computation unit on the prediction data input by the prediction data input unit to the prediction model generated by the prediction model generation unit.

5. The dementia prediction device according to claim 1, further comprising
a dimensional compression unit that performs predetermined dimensional compression processing on the relationship index value computed by the index value computation unit, thereby computing a dimensionally compressed relationship index value,
characterized in that the prediction model generation unit generates a prediction model for predicting the severity of the dementia based on a text index value group including a plurality of relationship index values for one text using a relationship index value dimensionally compressed by the dimensional compression unit, and
the dementia prediction unit applies a relationship index value obtained by further executing the processing of the dimensional compression unit on a relationship index value computed by the index value computation unit to the prediction model generated by the prediction model generation unit, thereby predicting the severity of the dementia for the patient subjected to prediction.

6. The dementia prediction device according to claim 1, characterized in that the prediction model generation unit computes a feature quantity associated with the severity of the dementia for the text index value group, and generates the prediction model for predicting the severity of the dementia from the text index value group based on the computed feature quantity.

7. The dementia prediction device according to claim 6, characterized in that the prediction model generation unit performs predetermined weighting calculation on the text index value group so that a value obtained by weighting calculation approaches a known value representing the severity of the dementia, and generates the prediction model for predicting the severity of the dementia from the text index value group using a weighted value for the text index value group as the feature quantity.

8. The dementia prediction device according to claim 1,
characterized in that the learning data input unit inputs, as learning data, a plurality of texts representing contents of free conversations conducted by a plurality of patients whose severity is known, respectively, for each of a plurality of evaluation items of the dementia,
the prediction model generation unit generates a prediction model for predicting severity for each of the evaluation items of the dementia based on the text index value group, and
the dementia prediction unit predicts the severity for each of the evaluation items of the dementia for the patient subjected to prediction.

9. The dementia prediction device according to claim 8, characterized in that the prediction model generation unit computes a feature quantity associated with severity for each of the evaluation items of the dementia for each of the evaluation items for the text index value group, and generates the prediction model for predicting severity for each of the evaluation items of the dementia from the text index value group based on the computed feature quantity.

10. The dementia prediction device according to claim 9, characterized in that the prediction model generation unit performs predetermined weighting calculation on the text index value group so that a value obtained by weighting calculation for each of the evaluation items approaches a known value representing the severity for each of the evaluation items of the dementia, and generates the prediction model for predicting the severity for each of the evaluation items of the dementia from the text index value group using a weighted value for the text index value group as the feature quantity for each of the evaluation items.

11. The dementia prediction device according to claim 1, characterized in that the severity of the dementia is a value of a score of a mini-mental state examination.

12. The dementia prediction device according to claim 1, characterized in that the severity of the dementia is a category classified by a number larger than 2 and less than a maximum value of the score of the mini-mental state examination.

13. A prediction model generation device characterized by comprising:
a learning data input unit that inputs a plurality of texts representing contents of free conversations conducted by a plurality of patients whose severity of dementia is known, respectively, as learning data;
an element extraction unit that analyzes morphemes of the plurality of texts input by the learning data input unit as the learning data, and extracts a plurality of decomposition elements from the plurality of texts;
a text vector computation unit that converts each of the plurality of texts into a q-dimensional vector (q is an arbitrary integer of 2 or more) according to a predetermined rule, thereby computing a plurality of text vectors including q axis components;
an element vector computation unit that converts each of the plurality of decomposition elements into a q-dimensional vector according to a predetermined rule, thereby computing a plurality of element vectors including q axis components;
an index value computation unit that obtains each of inner products of the plurality of text vectors and the plurality of element vectors, thereby computing a relationship index value reflecting a relationship between the plurality of texts and the plurality of decomposition elements; and
a prediction model generation unit that generates a prediction model for predicting the severity of the dementia based on a text index value group including a plurality of relationship index values for one text using the relationship index value computed by the index value computation unit.

14. The prediction model generation device according to claim 13,
characterized in that the learning data input unit inputs, as learning data, a plurality of texts representing contents of free conversations conducted by a plurality of patients whose severity is known, respectively, for each of a plurality of evaluation items of the dementia, and
the prediction model generation unit generates a prediction model for predicting severity for each of the evaluation items of the dementia based on the text index value group.

15. A dementia prediction device characterized by comprising:
a prediction data input unit that inputs one or more texts representing content of a free conversation conducted by a patient subjected to prediction as prediction data;
a second element extraction unit that analyzes morphemes of the one or more texts input by the prediction data input unit as the prediction data, and extracts a plurality of decomposition elements from the one or more texts;
a second text vector computation unit that converts the one or more texts into a q-dimensional vector (q is an arbitrary integer of 2 or more) according to a predetermined rule, thereby computing one or more text vectors including q axis components;
a second element vector computation unit that converts each of the plurality of decomposition elements into a q-dimensional vector according to a predetermined rule, thereby computing a plurality of element vectors including q axis components;
a second index value computation unit that obtains each of inner products of the one or more text vectors and the plurality of element vectors, thereby computing a relationship index value reflecting a relationship between the one or more texts and the plurality of decomposition elements; and
a dementia prediction unit that applies a relationship index value computed by the second index value computation unit to a prediction model generated by the prediction model generation device according to claim 13, thereby predicting the severity of the dementia for the patient subjected to prediction.

16. A dementia prediction program that causes a computer to function as:
learning data input means that inputs a plurality of texts representing contents of free conversations conducted by a plurality of patients whose severity of dementia is known, respectively, as learning data;
element extraction means that analyzes morphemes of the plurality of texts input by the learning data input means as the learning data, and extracts a plurality of decomposition elements from the plurality of texts;
text vector computation means that converts each of the plurality of texts into a q-dimensional vector (q is an arbitrary integer of 2 or more) according to a predetermined rule, thereby computing a plurality of text vectors including q axis components;
element vector computation means that converts each of the plurality of decomposition elements into a q-dimensional vector according to a predetermined rule, thereby computing a plurality of element vectors including q axis components;
index value computation means that obtains each of inner products of the plurality of text vectors and the plurality of element vectors, thereby computing a relationship index value reflecting a relationship between the plurality of texts and the plurality of decomposition elements; and
prediction model generation means that generates a prediction model for predicting the severity of the dementia based on a text index value group including a plurality of relationship index values for one text using the relationship index value computed by the index value computation means.

17. The dementia prediction program according to claim 16, further causing the computer to function as:
prediction data input means that inputs a text representing content of a free conversation conducted by a patient subjected to prediction as prediction data; and
dementia prediction means that predicts the severity of the dementia for the patient subjected to prediction by applying a relationship index value obtained by executing processes of the element extraction means, the text vector computation means, the element vector computation means, and the index value computation means on the prediction data input by the prediction data input means to the prediction model generated by the prediction model generation means.

18. A dementia prediction program that causes a computer to function as:
prediction data input means that inputs one or more texts representing content of a free conversation conducted by a patient subjected to prediction as prediction data;
second element extraction means that analyzes morphemes of the one or more texts input by the prediction data input means as the prediction data, and extracts a plurality of decomposition elements from the one or more texts;
second text vector computation means that converts the one or more texts into a q-dimensional vector (q is an arbitrary integer of 2 or more) according to a predetermined rule, thereby computing one or more text vectors including q axis components;
second element vector computation means that converts each of the plurality of decomposition elements into a q-dimensional vector according to a predetermined rule, thereby computing a plurality of element vectors including q axis components;
second index value computation means that obtains each of inner products of the one or more text vectors and the plurality of element vectors, thereby computing a relationship index value reflecting a relationship between the one or more texts and the plurality of decomposition elements; and
dementia prediction means that applies a relationship index value computed by the second index value computation means to a prediction model generated by the prediction model generation means according to claim 16, thereby predicting the severity of the dementia for the patient subjected to prediction.

19. The dementia prediction device according to claim 2, further comprising
a dimensional compression unit that performs predetermined dimensional compression processing on the relationship index value computed by the index value computation unit, thereby computing a dimensionally compressed relationship index value,
characterized in that the prediction model generation unit generates a prediction model for predicting the severity of the dementia based on a text index value group including a plurality of relationship index values for one text using a relationship index value dimensionally compressed by the dimensional compression unit, and
the dementia prediction unit applies a relationship index value obtained by further executing the processing of the dimensional compression unit on a relationship index value computed by the index value computation unit to the prediction model generated by the prediction model generation unit, thereby predicting the severity of the dementia for the patient subjected to prediction.

20. The dementia prediction device according to claim 3, further comprising
a dimensional compression unit that performs predetermined dimensional compression processing on the relationship index value computed by the index value computation unit, thereby computing a dimensionally compressed relationship index value,
characterized in that the prediction model generation unit generates a prediction model for predicting the severity of the dementia based on a text index value group including a plurality of relationship index values for one text using a relationship index value dimensionally compressed by the dimensional compression unit, and
the dementia prediction unit applies a relationship index value obtained by further executing the processing of the dimensional compression unit on a relationship index value computed by the index value computation unit to the prediction model generated by the prediction model generation unit, thereby predicting the severity of the dementia for the patient subjected to prediction.
